(19) United States
(12) Patent Application Publication
(10) Pub. No.: US 2007/0054814 A1
(43) Pub. Date: Mar. 8, 2007

(54) Lubricant composition, bearing apparatus, sliding member and triazine-ring compound

(75) Inventors: Masayuki Negoro, Ashigarakami-gun (JP); Ken Kawata, Ashigarakami-gun (JP); Hiroshi Kawamoto, Ashigarakami-gun (JP)

Correspondence Address: BUCHANAN, INGERSOLL & ROONEY PC, POST OFFICE BOX 1404, ALEXANDRIA, VA, 22313-1404, US

(73) Assignee: Fuji Photo Film Co., Ltd., Minami-ashigara-shi (JP)

(21) Appl. No.: 11/512,084

(22) Filed: Aug. 30, 2006

(30) Foreign Application Priority Data
Sep. 1, 2005 (JP) 2005/253189

(51) Int. Cl. C07D 251/54

(52) U.S. Cl. 508/258

(57) ABSTRACT

The novel lubricant composition is disclosed. The composition comprises at least one compound, exhibiting a minimum friction coefficient under a pressure equal to or greater than 10 MPa with the increase of a pressure and a viscosity-pressure coefficient equal to or less than 20 GPa⁻¹ at 40° C., represented by a formula (1).

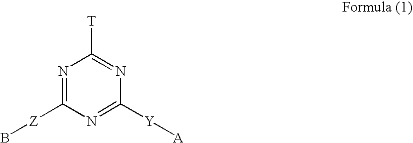

In the formula, Y and Z respectively represent a single bond or a bivalent linking group selected from the group consisting of NRa where Ra is a hydrogen atom or a C₁₋₃₀ alkyl group, oxygen, sulfur, carbonyl, sulfonyl and any combinations thereof; A and B respectively represent a substituted or non-substituted, alkyl group, alkenyl group, alkynyl group, aryl group or heterocyclic group; T is —S—R¹, —O—R² or —NR³R⁴; and R¹, R², R³ and R⁴ respectively represent a substituted or non-substituted, alkyl group, alkenyl group, alkynyl group, aryl group or heterocyclic group.

This application claims benefit of priority under 35 USC 119 to Japanese Patent Application No. 2005-253189 filed Sep. 1, 2005.

### BACKGROUND OF THE INVENTION

1. Field of the Invention

The present invention relates to a lubricant composition to be supplied to mechanical friction sliding parts, and, in particular, to a lubricant composition not only exhibiting an excellent low-friction property and an excellent wear-resistance under an extreme-pressure but also exhibiting such properties for a long term. And the present invention relates to an apparatus employing the lubricant composition in the sliding part, to a sliding member impregnated with the composition. The present invention also relates to a triazine-ring compound which is useful as lubricant.

2. Related Art

Performances required for lubricant relate to that it should be able to lower friction coefficient at mechanical friction sliding parts over a wide temperature range and pressure range, and that such effects are sustained as long as possible. It is also expected for the lubricant to not only improve lubricating properties between mechanical friction sliding parts, but also to thereby provide wear resistance to such friction sliding members in themselves. Effects, which are obtainable by using lubricant such as engine oil, of reducing friction coefficient of the friction sliding parts and increasing service life thereof directly result in improved fuel cost for mechanical driving, or in other words, energy saving. Extension of the service life of engine oil not only ensures reduction in waste oil but also reduction in CO₂ emission, so that it will be desirable in terms of environmental compatibility which has increasingly been attracting recent public attention. As for bearings or gears, which operate under particularly severe frictional conditions among various sliding parts for use in industrial machines, use of conventional lubricant such as lubricating oil or grease may result in film breakage or sticking of the lubricant under particularly severe lubricating conditions, which makes it difficult to obtain a desired low friction coefficient due to abrasion scars. This sometimes lowers the reliability of apparatus, and tends to increase severity of the friction conditions especially for the case that the apparatus is to be downsized, which has been one reason for preventing the apparatus from being downsized. So that there has been a strong demand for a lubricant which can bring about the effects even under severe conditions, can contribute to downsizing of the apparatus, and is excellent in energy saving property.

Lubricants which have previously been used are generally such that comprising lubricant base oil as a major component, and a lubricant-auxiliary agent such as an organic compound blended thereto. In particular, organic molybdenum compounds recently have attracted an attention as a lubricant-auxiliary agent. Organic molybdenum compounds are excellent in various properties such as wear resistance, durability under extreme pressure (load resistance) and low friction property even during operation of sliding parts of a mechanical apparatus under severe frictional conditions such as high temperature, high or low speed, high load, downsizing and weight reduction, so that the compounds have attracted a good deal of attention as a material capable of effectively exhibiting lubricating effects under a marginal lubricating condition which is higher in pressure than the fluid lubricating condition under ordinary pressure.

Although the organic molybdenum compound may exhibit an excellent lubricant effect even under a severe friction condition, it is apparently inappropriate in view of environmental compatibility since the lubricating oil contains a considerable amount of heavy metals such as molybdenum and zinc, sulfide which can readily be oxidized to thereby produce sulfur oxide adversely affecting the lubricating oil or sliding part per se, and even affecting the environment, and phosphoric acid which undesirably eutrophicates rivers and seas. Another disadvantage relates to that molybdenum oxide/sulfide film formed on the sliding surface is gradually peeled off under friction to thereby produce a new film, so that shortage in the amount of either of organic molybdenum compound or organic zinc compound, which are source materials, may sharply lose the effect. A countermeasure of increasing the amount of such organic molybdenum compound and organic zinc compound is however undesirable since it may increase the amount of byproducts generated in the system by such peeling-off of the film, which adversely affect the sliding machinery per se, so that it is less expectable in a current situation of a system using the foregoing organic molybdenum compound to improve fuel cost through elongation of the service life of the lubricant. As has been described in the above, there has been no proposal of a lubricant which is free from any of environmentally hazardous substance or environmental pollutant such as heavy metal elements, phosphate compounds and sulfides, capable of exhibiting excellent lubricating properties, and capable of retaining such properties for a long period.

As described above, lubricants without any environmental toxins or pollutants such as heavy metal elements, phosphoric acid compounds and sulfide compounds, not only exhibiting excellent lubricating properties but also exhibiting such properties for a long term, have not been provided yet.

It has been known that a lubricant composition comprising a triazine-ring-containing compound as a major component has an excellent environmental compatibility or can contribute to improvement of fuel consumption due to long-life property, and that the composition exhibits properties enough to be as an extreme pressure agent, friction-coefficient-lowering agent and anti-wear additives (see Japanese Laid-Open Patent Publication No. 2002-69472).

Lubricants have been recently required to have more various properties and higher performances with the developments of various high performance machines and with frequent use under severe conditions.

And it has been strongly required, along with the development of high-performance AV and OA apparatuses or along with the popularization of mobile use, to improve small spindle motors to be employed in rotating members in the view of speed-up or dwinsizeing. And, therefore, it has been also required to improve bearings to be employed in members for supporting rotating members in the view of low torque property. Various factors such as bearing clearances and diameters of spindles affect on torque of a bearing, and one important factor among them is viscosity of lubricant. In usual, it is known that lubricant oils having lower viscosities tend to vaporize more readily. When lubricant oils are lost due to vaporization or the like, appropriate oil films can not be formed. It may result in lowering the rotation accuracy by which the lifetime is determined. And, thus, the vaporization property of a lubricant oil is one important property which influence the durability of the lubricant oil. Accordingly, it is necessary to select lubricant in the view of low viscosity and good vaporization property, for lubricating slide bearings such as fluid dynamical pressure bearings, impregnated porous bearings and dynamical pressure type impregnated porous bearings, in the view of low viscosity and good vaporization property.

### SUMMARY OF THE INVENTION

One object of the present invention is to provide a lubricant composition capable of exhibiting excellent properties not only in a state of mixture with conventional lubricant base oil, but also in a state not mixed with such lubricant base oil, and a novel triazine-ring compound useful for the composition.

Another object of the present invention is to provide a lubricant composition capable of retaining low friction property and antiwear on the sliding surface for a long period, in particular even under extreme pressure, and a novel triazine-ring compound useful for the composition.

Another object of the present invention is to provide a lubricant composition without environmentally-less-compatible heavy metals, phosphate group and sulfides to thereby concomitantly achieve both of longer service life and environmental compatibility, and a novel triazine-ring compound useful for the composition.

Another object of the present invention is to provide a lubricant composition, exhibiting a low viscosity, excellent in evaporation characteristic, and a novel triazine-ring compound useful for the composition.

Another object of the invention is to provide a bearing apparatus which is long-life and is capable or working stably, and to provide a sliding member useful for a bearing apparatus.

Under the above circumstances, the present inventors conducted various studies in order to solve the problems, and as a result, they found that, a particular class of compounds exhibit excellent lubricating properties. On the basis of these findings, the present invention was made.

In one aspect, the invention provides a lubricant composition comprising at least one compound, exhibiting a minimum friction coefficient under a pressure equal to or greater than 10 MPa with the increase of a pressure and a viscosity-pressure coefficient equal to or less than 20 GPa⁻¹ at 40° C., represented by a formula (1):

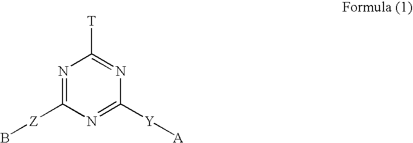

where Y and Z respectively represent a single bond or a bivalent linking group selected from the group consisting of NRa where Ra is a hydrogen atom or a C₁₋₃₀ alkyl group, oxygen, sulfur, carbonyl, sulfonyl and any combinations thereof; A and B respectively represent a substituted or non-substituted, alkyl group, alkenyl group, alkynyl group, aryl group or heterocyclic group; T is —S—R¹, —O—R² or —NR³R⁴; and R¹, R², R³ and R⁴ respectively represent a substituted or non-substituted, alkyl group, alkenyl group, alkynyl group, aryl group or heterocyclic group.

The lubricant composition may exhibit apparent viscosity not more than 300 mPa·S at 40° C.

The compound represented by the formula (1) may be selected from the group represented by a formula (2):

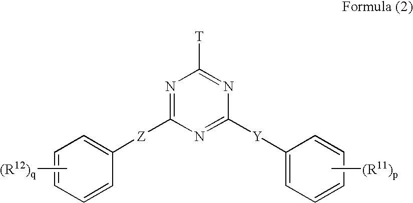

where Y and Z respectively represent a single bond or a bivalent linking group selected from the group consisting of NRa where Ra is a hydrogen atom or a C₁₋₃₀ alkyl group, oxygen, sulfur, carbonyl, sulfonyl and any combinations thereof; R¹¹ and R¹² respectively represent a substituent; T is —S—R¹, —O—R² or —NR³R⁴; R¹R², R³ and R⁴ respectively represent a substituted or non-substituted, alkyl group, alkenyl group, alkynyl group, aryl group or heterocyclic group; and p and q respectively represents an integer from 1 to 5.

As embodiments of the invention, there are provided the lubricant composition wherein, in the formula (1), both of Y and z are sulfur atoms; the lubricant composition wherein, in the formula (1), both of Y and z are oxygen atoms; and the lubricant composition wherein, in the formula (1), at least one of T, A and B contains an oligoalkyleneoxy group.

The lubricant composition may be used as an impregnating oil composition for a sintered bearing.

In another aspect, the invention provides a sliding member comprising a sintered body impregnated with a composition of the invention; and a bearing apparatus for bearing a rotating element rotatably comprising a sliding part wherein at least a part of the sliding part is a sintered body impregnated with the composition of the invention.

In another aspect, the invention provides a triazine-ring compound represented by the formula (2).

### PREFERRED EMBODIMENT OF THE INVENTION

The present invention will be described in detail. It is to be understood, in this description, that the term “ . . . to . . . ” is used as meaning a range inclusive of the lower and upper values disposed therebefore and thereafter.

The lubricant composition of the invention comprises at least one compound represented by a formula (1) described below.

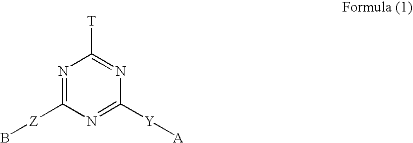

In the formula, Y and Z respectively represent a single bond or a bivalent linking group selected from the group consisting of NRa where Ra is a hydrogen atom or a C₁₋₃₀ alkyl group, oxygen, sulfur, carbonyl, sulfonyl and any combinations thereof; A and B respectively represent a substituted or non-substituted, alkyl group, alkenyl group, alkynyl group, aryl group or heterocyclic group; T is —S—R¹, —O—R² or —NR³R⁴; and R¹, R², R³ and R⁴ respectively represent a substituted or non-substituted, alkyl group, alkenyl group, alkynyl group, aryl group or heterocyclic group.

In the formula, Y and Z respectively represent a single bond or a bivalent linking group selected from the group consisting of NRa where Ra is a hydrogen atom or a C₁₋₃₀ linear or branched alkyl group, oxygen, sulfur, carbonyl, sulfonyl and any combinations thereof. Examples of the bivalent linking group include oxycarbonyl (—OC(═O)—), aminocarbonyl (—NHC(═O)—), carbamoyl (—C(═O)NH—), oxysulfonyl (—OSO₂—) and sulfamoyl (—SO₂NH—) In the case that Y or Z is a single bond, it binds to the triazine ring in the formula (1) directly. In the case that Y or Z is a single bond and A or B is a heterocyclic group, Y and A or Z and B may bind directly through nitrogen atom, having free atomic valence, of the heterocyclic group, A or B, such as a piperidine residue, or may bind directly through a heteroatom not having free atomic valence to form an onium salt such as an oxonium salt, sulfonium salt or ammonium salt. It is preferred that Y and Z respectively represent a sulfur atom or an oxygen atom; and it is more preferred that both of Y and Z are sulfur atoms or oxygen atoms.

In the formula, A and B respectively represent a substituted or non-substituted, alkyl group, alkenyl group, alkynyl group, aryl group or heterocyclic group. The carbon atom number of the alkyl group represented by A or B is preferably from 1 to 30, more preferably from 2 to 30, much more preferably from 4 to 30, and further much more preferably from 6 to 30. The alkyl group may have a linear or branched chain structure, and may have one or more substituents. Examples of the substituent include halogen atoms, alkoxy groups such as methoxy, ethoxy, methoxyethoxy and phenoxy; sulfide groups such as methylthio, ethylthio and propylthio; alkylamino groups such as methylamino and propylamino; acyl groups such as acetyl, propanoyl, octanoyl and benzoyl; acyloxy groups such as acetoxy, pivaloyloxy and benzoyloxy; hydroxyl, mercapto, amino, cyano, carboxyl, sulfo, carbamoyl, sulfamoyl and ureido.

The carbon atom number of the alkenyl or alkynyl group represented A or B is preferably from 2 to 30, more preferably from 3 to 30, much more preferably from 4 to 30 and further much more preferably from 6 to 30. The alkenyl or alkynyl group may have a linear or branched chain structure, and may have one or more substituents. Examples of the substituent are same as those exemplified as a substituent for the alkyl group.

The aryl group represented by A or B may contain a single ring or a condensed ring formed of two or more rings. Preferred examples of the aryl group include phenyl, indenyl, alpha-naphthyl, beta-naphthyl, fluorenyl, phenanthryl, anthracenyl and pyrenyl. Phenyl and naphthyl are more preferred. The aryl group may have one or more substituents. Examples of the substituent include alkyl groups and those exemplified above as substituents of the alkyl group. It is preferred that the aryl group has one or more substituents including C₈ or longer linear or branched alkyls and substituents containing a C₈ or longer linear or branched alkyl residue. Examples of the C₈ or longer linear or branched alkyl group include octyl, decyl, hexadecyl and 2-ethylhexyl. Examples of the substituent containing a C₈ or longer linear or branched alkyl residue include alkoxy groups such as dodecyloxy and hexadecyloxy; sulfide groups such as hexadecylthio; substituted amino groups such as heptadecyl amino, octyl carbamoyl, octanoyl and decyl sulfamoyl. The aryl group preferably has two or more substituents selected from the substituents including a C₈ or longer linear or branched alkyl residue. The aryl group may have one or more substituents selected from other substituents such as halogen atoms, hydroxyl, cyano, nitro, carboxyl and sulfo.

The heterocyclic group represented by A or B is preferably selected from 5-, 6- or 7-membered heterocyclic groups, more preferably selected from 5- or 6-membered heterocyclic groups, and much more preferably selected from 6-membered heterocyclic groups. The heterocyclic group may contain a single ring or a condensed ring formed of two or more rings. Specific examples of such skeletons can be found in heterocycles listed in “Iwanami Rikagaku Jiten (Iwanami's Physicochemical Dictionary; Iwanami Shoten, Publishers), the 3rd edition, supplement Chapter 11 “Nomenclature for Organic Chemistry”, Table 4 “Names of Principal Hetero Monocyclic Compounds” on page 1606, and Table 5 “Names of Principal Condensed Heterocyclic Compounds” on page 1607. The heterocyclic groups are, similarly to the foregoing aryl group, preferably substituted with a substituent containing a C₈ or longer linear or branched alkyl chain, where substitution by two or more groups is more preferable. Specific examples of the substituent including such chain are same as those described in the above. The heterocyclic group may also be substituted by halogen atom, hydroxyl, cyano, nitro, carboxyl, sulfo or the like, besides the foregoing substituents.

It is preferred that A and B contain at least one, linear or branched alkyl chain having the total number of carbon atoms embedded therein equal to or greater than 8; linear or branched oligoalkyleneoxy chain having the total number of carbon atoms embedded therein ranging from 4 to 48, linear or branched perfluoroalkyl chain having the total number of carbon atoms embedded therein equal to or more than 2; linear or branched perfluoroalkylether chain having the total number of carbon atoms embedded therein equal to or greater than 2; or linear or branched organic polysiloxyl chain. It is more preferred that A and B are phenyl groups having at least one linear or branched oligoalkyleneoxy chain, preferably having the total number of carbon atoms embedded therein ranging from 4 to 48. The total number of carbon atoms embedded in the oligoalkyleneoxy chain more preferably ranges from 4 to 24. Preferred examples of the alkylene group embedded in the oligoalkyleneoxy-chain include ethylene, propylene and butylene. The number of the alkyleneoxy group embedded in the oligoalkyleneoxy chain preferably ranges from 2 to 7 and more preferably ranges from 2 to 5.

In the formula, T is —S—R¹, —O—R² or —NR³R⁴. R¹, R², R³ and R⁴ respectively represent a substituted or non-substituted, alkyl group, alkenyl group, alkynyl group, aryl group or heterocyclic group.

The alkyl group represented by R¹, R², R³ or R⁴ is preferably selected from C₁₋₃₀ alkyl groups, more preferably selected from C₂₋₃₀ alkyl groups, much more preferably selected from C₄₋₃₀ alkyl groups, and further much more preferably selected from C₆₋₃₀ alkyl groups. The alkyl group may have a linear or branched chain structure. And the alkyl group may have one or more substituents. Examples of the substituent include halogen atoms, C₁₋₄₀, preferably C₁₋₂₀, alkoxy groups such as methoxy, ethoxy, methoxyethoxy and phenoxy; C₁₋₄₀, preferably C₂₀, alkylthio groups and C₆₋₄₀, preferably C₆₋₂₀, alylthio groups such as methylthio, ethylthio and propylthio; C₁₋₄₀, preferably C₁₋₂₀, alkylamino groups such as methylamino and propylamino; C₁₋₄₀, preferably C₁₋₂₀, acyl groups such as acetyl, propanoyl, octanoyl and benzoyl; C₁₋₄₀, preferably C₂₋₂₀, acyloxy groups such as acetoxy, pivaloyloxy and benzoyloxy; hydroxyl, mercapto, amino, carboxyl, sulfo, carbamoyl, sulfamoyl and ureido.

The alkenyl or alkynyl group represented by R¹, R², R³ or R⁴ is preferably selected from C₂₋₃₀ alkenyl or alkynyl groups, more preferably selected from C₃₋₃₀ alkenyl or alkynyl groups, much more preferably selected from C₄₋₃₀ alkenyl or alkynyl groups and further much more preferably selected from C₆₋₃₀ alkenyl or alkynyl groups. The alkenyl or alkynyl group may have a linear or branched chain structure. The alkenyl or alkynyl group may have one or more substituents selected from the groups exemplified above as substituents of the alkyl group.

The aryl group represented by R¹, R², R³ or R⁴ may contain a single ring or a condensed ring formed of two or more rings. It is preferred that R¹, R², R³ or R⁴ is phenyl, indenyl, alpha-naphthyl, beta-naphthyl, fluorenyl, phenanthryl, anthracenyl or pyrenyl, and it is more preferred that it is phenyl or naphthyl. The aryl group may have one or more substituents. Examples of the substituent include alkyl groups and those exemplified above as substituents of the alkyl group. It is preferred that the aryl group has one or more substituents containing a C₈ or longer linear or branched alkyl residue, such as alkyl groups (e.g. octyl, decyl, hexadecyl and 2-ethylhexyl); alkoxy groups (e.g. dodecyloxy and hexadecyloxy); sulfide groups (e.g. hexadecylthio); substituted amino groups (e.g. heptadecyl amino), octyl carbamoyl, octanoyl and decyl sulfamoyl. The aryl group preferably has two or more substituents selected from the substituents containing a C₈ or longer linear or branched alkyl residue. The aryl group may have one or more substituents selected from other substituents such as halogen atoms, hydroxyl, cyano, nitro, carboxyl and sulfo.

The heterocyclic group represented by R¹, R², R³ or R⁴ is preferably selected from 5-, 6- or 7-membered heterocyclic groups, more preferably selected from 5- or 6-membered heterocyclic groups, and much more preferably selected from 6-membered heterocyclic groups. The heterocyclic group may contain a single ring or a condensed ring formed of two or more rings. Specific examples of such skeletons can be found in hetero rings listed in “Iwanami Rikagaku Jiten (Iwanami's Physicochemical Dictionary; Iwanami Shoten, Publishers), the 3rd edition, supplement Chapter 11 “Nomenclature for Organic Chemistry”, Table 4 “Names of Principal Hetero Monocyclic Compounds” on page 1606, and Table 5 “Names of Principal Condensed Heterocyclic Compounds” on page 1607. The heterocyclic groups are, similarly to the foregoing aryl group, preferably substituted with a substituent containing a C₈ or longer linear or branched alkyl chain, where substitution by two or more groups is more preferable. Specific examples of the substituent including such chain are same as those described in the above. The heterocyclic group may also be substituted by halogen atom, hydroxyl, cyano, nitro, carboxyl, sulfo or the like, besides the foregoing substituents.

It is preferred that R¹, R², R³ or R⁴ contains at least one, linear or branched alkyl chain having the total number of carbon atoms equal to or greater than 8; linear or branched oligoalkyleneoxy chain having the total number of the carbon atoms embedded therein ranging from 4 to 48 (the preferred scope of the oligoalkyleneoxy chain is same as described above); linear or branched perfluoroalkyl chain having the total number of carbon atoms equal to or greater than 2; linear or branched perfluoroalkylether chain having the total number of carbon atoms equal to or greater than 2; or linear or branched organic polysiloxyl chain.

The compound represented by the formula (1) is preferably selected from the groups represented by a formula (2).

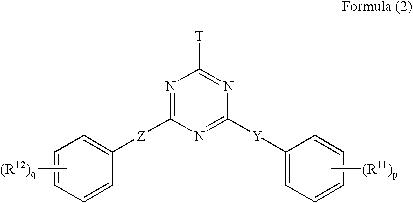

In the formula (2), the definitions and preferred examples of Y, Z and T are same as those in the formula (1). R¹¹” and R¹² respectively represent a substituent; and p and q respectively represents an integer from 1 to 5.

Examples of the substituent represented by R¹¹ or R¹² include linear or branched alkyl groups, halogen atoms, hydroxyl, cyano, nitro, carboxyl and sulfo. It is preferred that R¹¹ and R¹² respectively represent a substituent containing a linear or branched alkyl chain having the total number of carbon atoms embedded therein equal to or greater than 8; a linear or branched oligoalkyleneoxy chain having the total number of carbon atoms embedded therein ranging from 4 to 48; a linear or branched perfluoroalkyl chain having the total number of carbon atoms embedded therein equal to or greater than 2; a linear or branched perfluoroalkylether chain having the total number of carbon atoms equal to or greater than 2; or a linear or branched organic polysiloxyl chain. It is more preferred that R¹¹ and R¹² respectively represent a substituent containing a linear or branched oligoalkyleneoxy chain having the total carbon atoms embedded therein ranging from 4 to 48. The preferred scope of the oligoalkyleneoxy chain is same as described above.

Preferred examples of the compound represented by the formula (2) include the compounds wherein both of Y and Z are sulfur atoms, and wherein both of Y and Z are oxygen atoms. Preferred examples of the compound represented by the formula (2) also include the compounds wherein at least one of T, R¹¹ and R¹² represents a substituent containing an oligoalkyleneoxy chain. The preferred scope of the oligoalkyleneoxy chain is same as described above.

Examples of the compound represented by the formula (1) include, but are not limited to, those shown below.

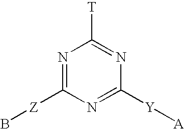

| | —Y—A | —Z—B | T |
|---|---|---|---|
| S-1 | 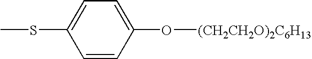 | 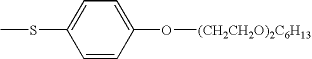 | —OCH₃ |
| S-2 | 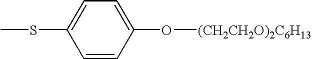 | 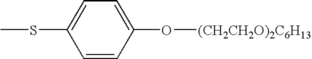 | —OC₂H₅ |
| S-3 | 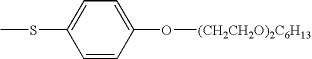 | 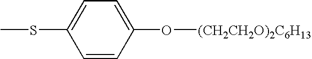 | —OC₃H₇ |
| S-4 | 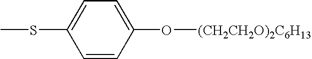 | 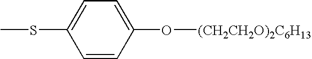 | —OC₆H₁₃ |
| S-5 | 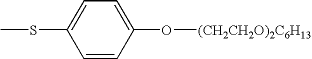 | 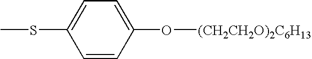 | —OC₁₂H₂₅ |
| S-6 | 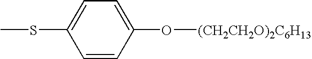 | 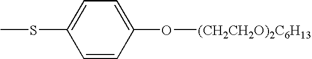 | 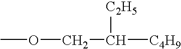 |
| S-7 | 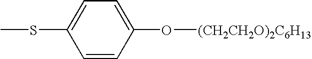 | 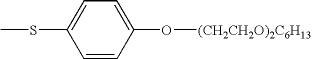 | —O—(CH₂CH₂O)₂C₆H₁₃ |
| S-8 | 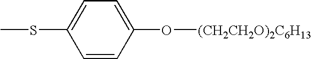 | 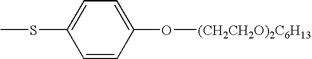 | —O—(CH₂CH₂O)₃CH₃ |
| S-9 | 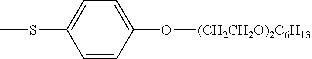 | 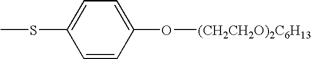 | —O—CH₂CF₂(OCF₂CF₂)₂OC₃F₇ |
| S-10 | 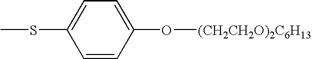 | 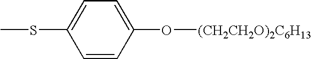 | —O—CH₂(OCF₂)₂(OCF₂CF₂)₂CF₃ |
| S-11 | 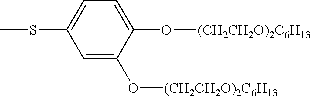 | 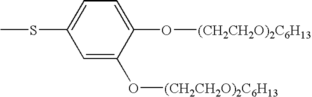 | —OCH₃ |
| S-12 | 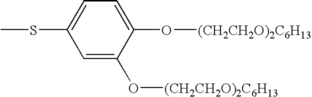 | 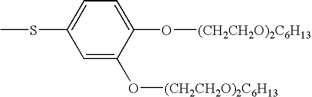 | —OC₂H₅ |
| S-13 | 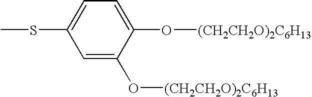 | 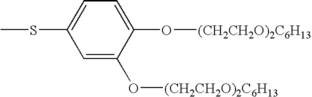 | —OC₃H₇ |
| S-14 | 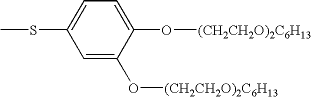 | 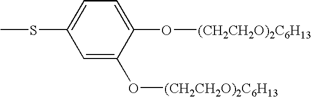 | —OC₆H₁₃ |
| S-15 | 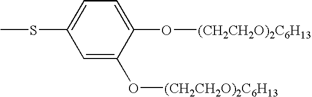 | 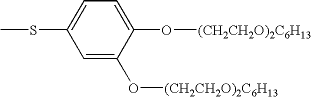 | —OC₁₂H₂₅ |
| S-16 | 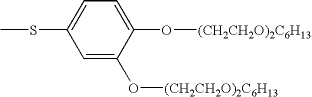 | 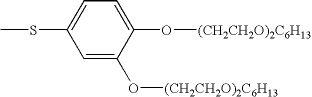 | 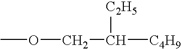 |
| S-17 | 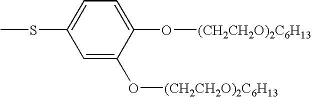 | 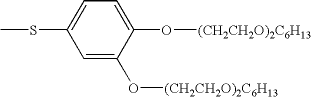 | —O—(CH₂CH₂O)₂C₆H₁₃ |
| S-18 | 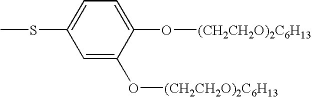 | 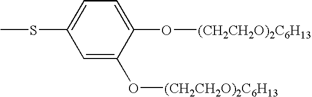 | —O—(CH₂CH₂O)₃CH₃ |
| S-19 | 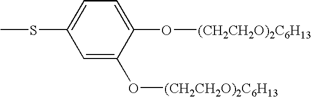 | 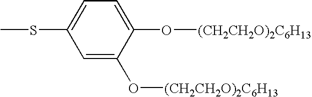 | —O—CH₂CF₂(OCF₂CF₂)₂OC₃F₇ |
| S-20 | 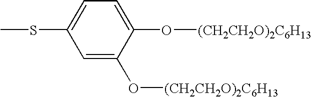 | 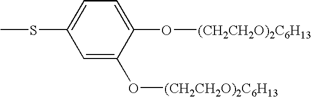 | —O—CH₂(OCF₂)₂(OCF₂CF₂)₂CF₃ |
| S-21 | 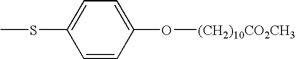 | 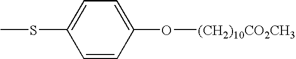 | —OCH₃ |
| S-22 | 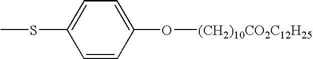 | 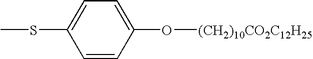 | —OC₂H₅ |
| S-23 | 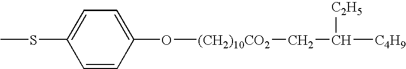 | 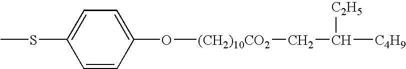 | —OC₃H₇ |
| S-24 | 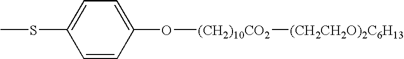 | 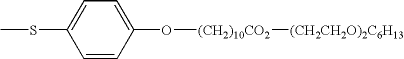 | —OC₆H₁₃ |
| S-25 | 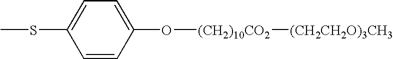 | 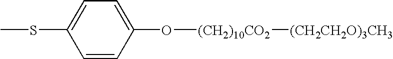 | —OC₁₂H₂₅ |
| S-26 | 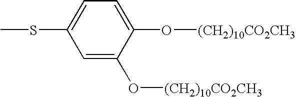 | 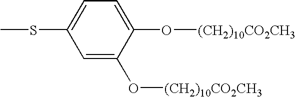 | 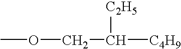 |
| S-27 | 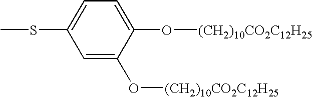 | 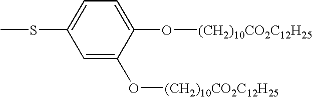 | —O—(CH₂CH₂O)₂C₆H₁₃ |
| S-28 | 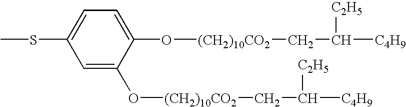 | 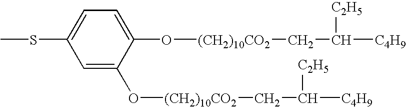 | —O—(CH₂CH₂O)₃CH₃ |
| S-29 | 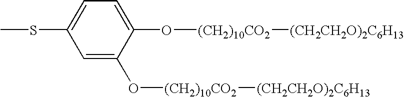 | 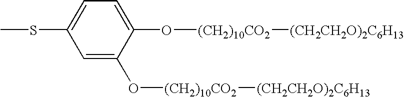 | —O—CH₂CF₂(OCF₂CF₂)₂OC₃F₇ |
| S-30 | 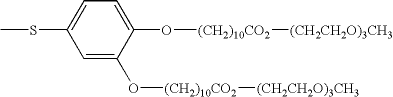 | 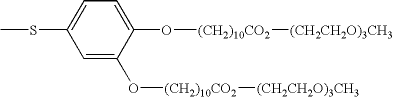 | —O—CH₂(OCF₂)₂(OCF₂CF₂)₂CF₃ |
| S-31 | 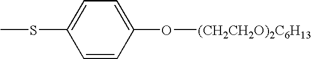 | 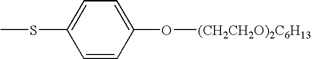 | 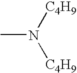 |
| S-32 | 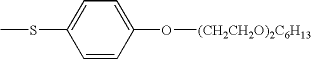 | 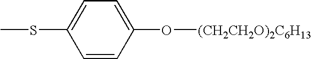 | 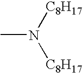 |
| S-33 | 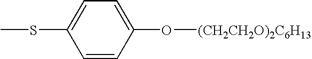 | 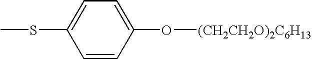 | 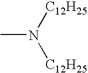 |
| S-34 | 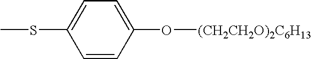 | 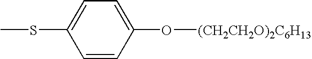 | 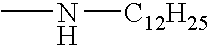 |
| S-35 | 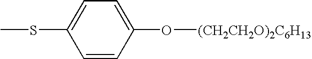 | 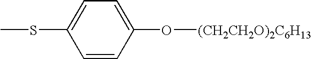 | 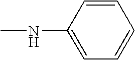 |
| S-36 | 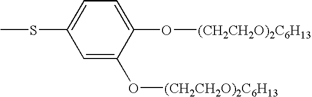 | 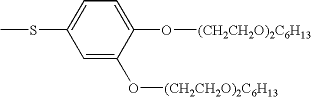 | 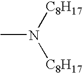 |
| S-37 | 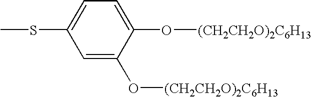 | 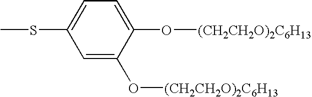 | 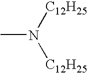 |
| S-38 | 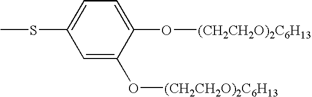 | 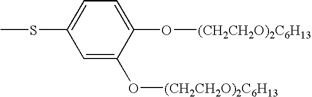 | 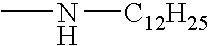 |
| S-39 | 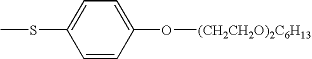 | 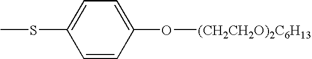 | —SC₁₂H₂₅ |
| S-40 | 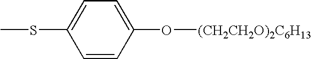 | 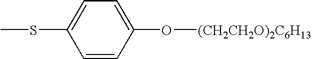 | 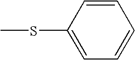 |
| S-41 | 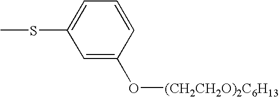 | 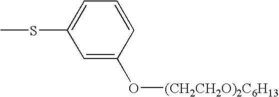 | —O—(CH₂CH₂O)₂C₆H₁₃ |
| S-42 | 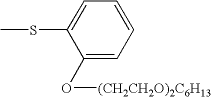 | 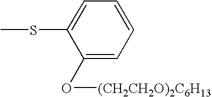 | —O—(CH₂CH₂O)₂C₆H₁₃ |
| S-43 | 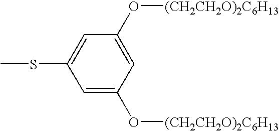 | 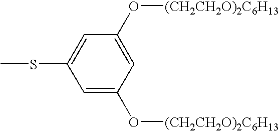 | —O—(CH₂CH₂O)₂C₆H₁₃ |
| S-44 | 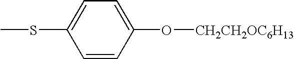 | 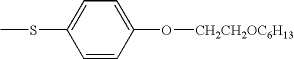 | —O—CH₂CH₂OC₆H₁₃ |
| S-45 | 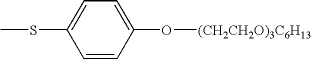 | 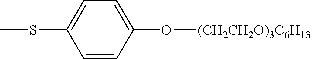 | —O—(CH₂CH₂O)₃C₆H₁₃ |
| S-46 | 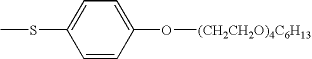 | 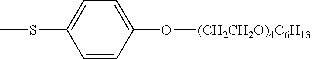 | —O—(CH₂CH₂O)₄C₆H₁₃ |
| S-47 | 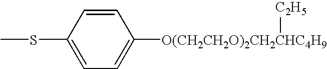 | 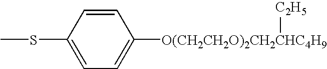 | —O—(CH₂CH₂O)₂C₆H₁₃ |
| S-48 | 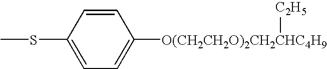 | 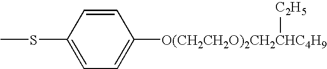 | 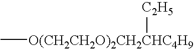 |
| S-49 | 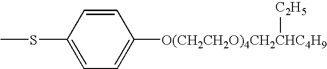 | 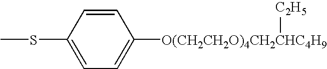 | 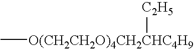 |
| S-50 | 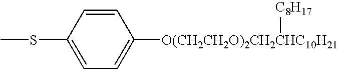 | 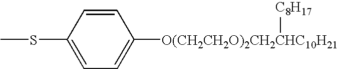 | —O—(CH₂CH₂O)₄C₆H₁₃ |
| O-1 | 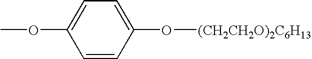 | 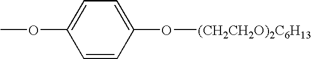 | —OCH₃ |
| O-2 | 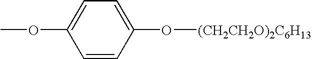 | 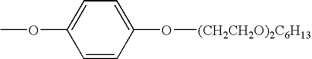 | —OC₂H₅ |
| O-3 | 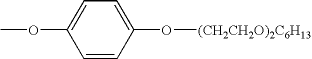 | 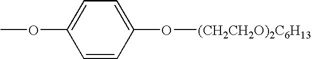 | —OC₃H₇ |
| O-4 | 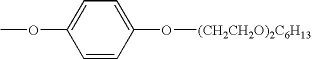 | 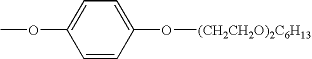 | —OC₆H₁₃ |
| O-5 | 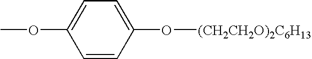 | 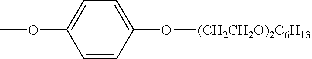 | —OC₁₂H₂₅ |
| O-6 | 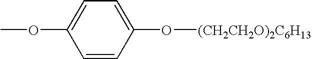 | 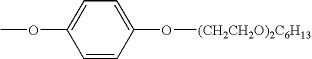 | 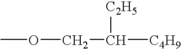 |
| O-7 | 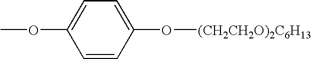 | 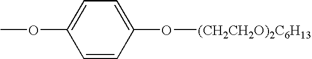 | —O—(CH₂CH₂O)₂C₆H₁₃ |
| O-8 | 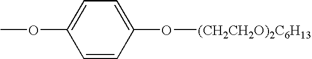 | 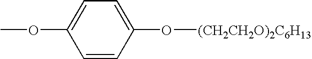 | —O—(CH₂CH₂O)₃CH₃ |
| O-9 | 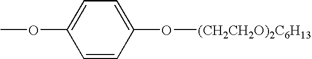 | 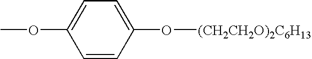 | —O—CH₂CF₂(OCF₂CF₂)₂OC₃F₇ |
| O-10 | 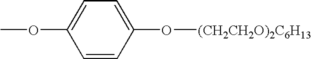 | 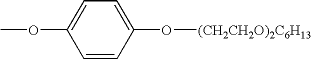 | —O—CH₂(OCF₂)₂(OCF₂CF₂)₂CF₃ |
| O-11 | 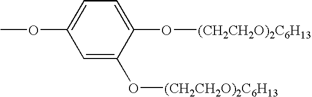 | 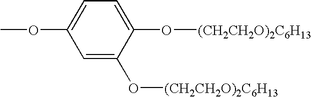 | —OCH₃ |
| O-12 | 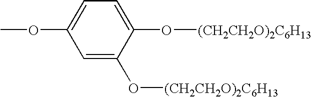 | 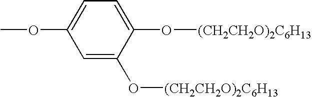 | —OC₂H₅ |
| O-13 | 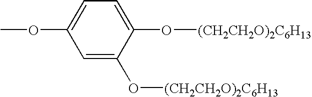 | 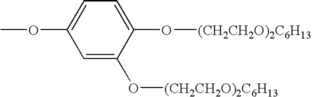 | —OC₃H₇ |
| O-14 | 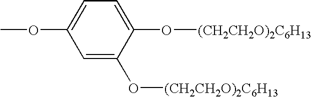 | 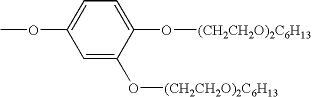 | —OC₆H₁₃ |
| O-15 | 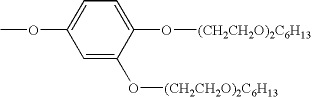 | 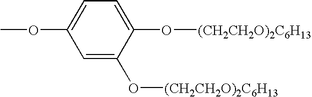 | —OC₁₂H₂₅ |
| O-16 | 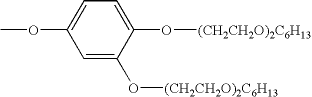 | 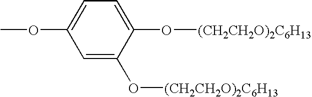 | 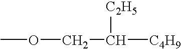 |
| O-17 | 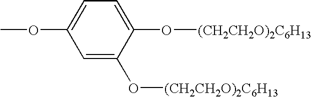 | 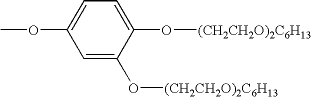 | —O—(CH₂CH₂O)₂C₆H₁₃ |
| O-18 | 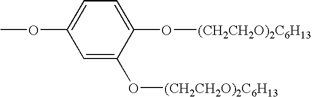 | 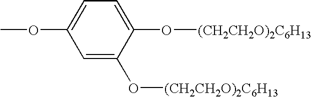 | —O—(CH₂CH₂O)₃CH₃ |
| O-19 | 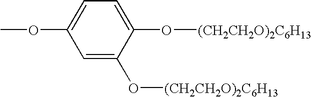 | 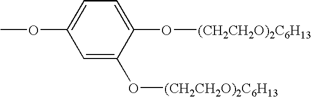 | —O—CH₂CF₂(OCF₂CF₂)₂OC₃F₇ |
| O-20 | 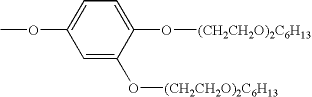 | 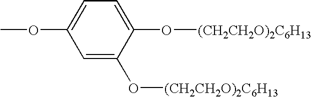 | —O—CH₂(OCF₂)₂(OCF₂CF₂)₂CF₃ |
| O-21 | 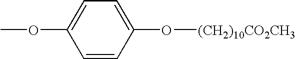 | 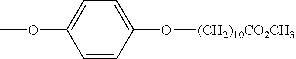 | —OCH₃ |
| O-22 | 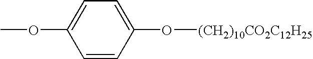 | 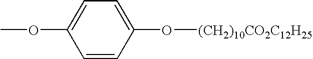 | —OC₂H₅ |
| O-23 | 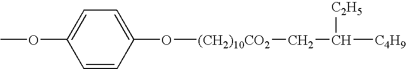 | 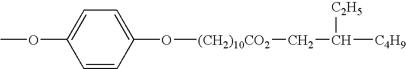 | —OC₃H₇ |
| O-24 | 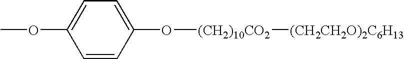 | 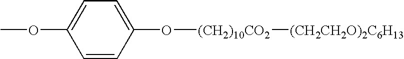 | —OC₆H₁₃ |
| O-25 | 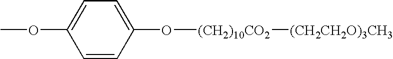 | 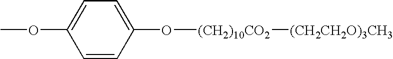 | —OC₁₂H₂₅ |
| O-26 | 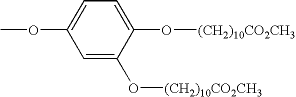 | 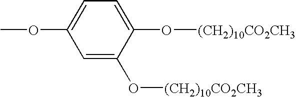 | 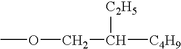 |
| O-27 | 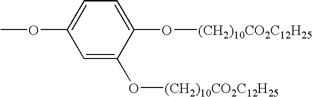 | 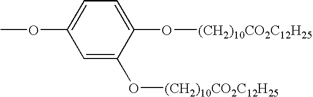 | —O—(CH₂CH₂O)₂C₆H₁₃ |
| O-28 | 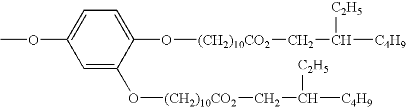 | 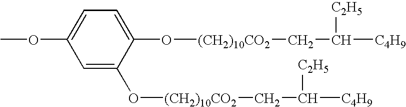 | —O—(CH₂CH₂O)₃CH₃ |
| O-29 | 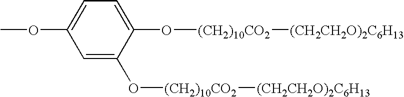 | 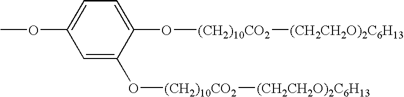 | —O—CH₂CF₂(OCF₂CF₂)₂OC₃F₇ |
| O-30 | 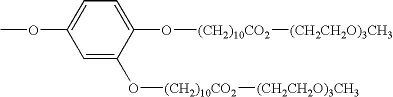 | 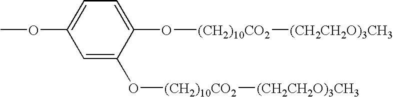 | —O—CH₂(OCF₂)₂(OCF₂CF₂)₂CF₃ |
| O-31 | 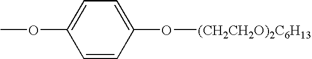 | 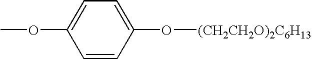 | 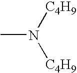 |
| O-32 | 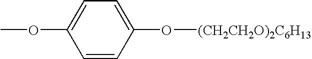 | 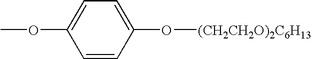 | 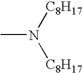 |
| O-33 | 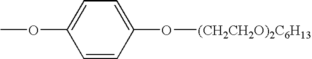 | 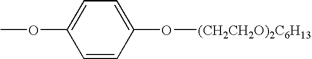 | 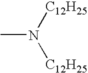 |
| O-34 | 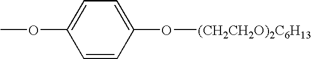 | 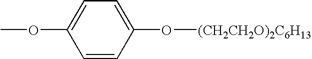 | 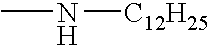 |
| O-35 | 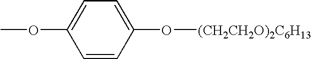 | 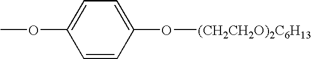 | 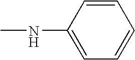 |
| O-36 | 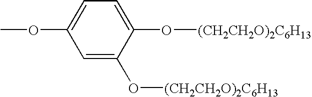 | 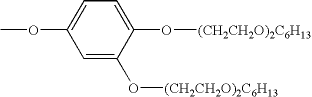 | 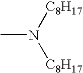 |
| O-37 | 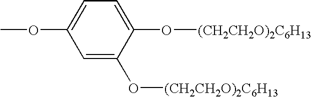 | 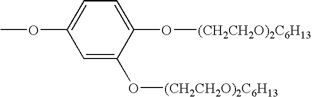 | 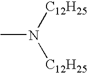 |
| O-38 | 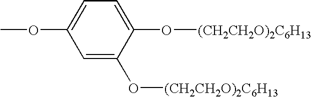 | 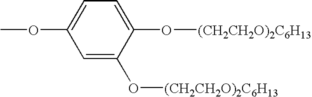 | 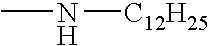 |
| O-39 | 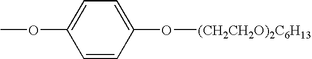 | 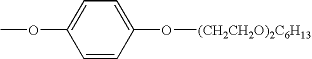 | —SC₁₂H₂₅ |
| O-40 | 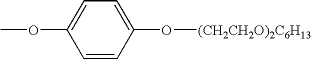 | 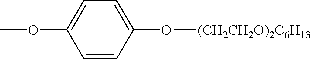 | 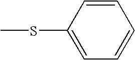 |
| O-41 | 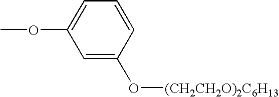 | 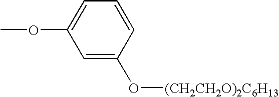 | —O—(CH₂CH₂O)₂C₆H₁₃ |
| O-42 | 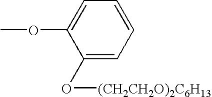 | 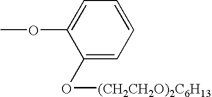 | —O—(CH₂CH₂O)₂C₆H₁₃ |
| O-43 | 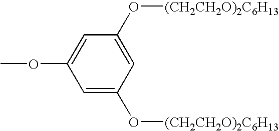 | 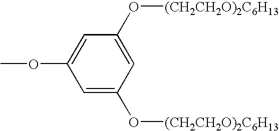 | —O—(CH₂CH₂O)₂C₆H₁₃ |
| O-44 | 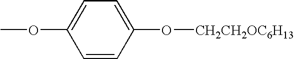 | 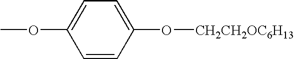 | —O—CH₂CH₂OC₆H₁₃ |
| O-45 | 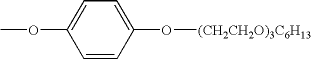 | 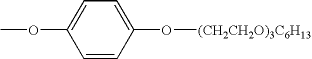 | —O—(CH₂CH₂O)₃C₆H₁₃ |
| O-46 | 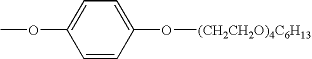 | 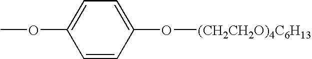 | —O—(CH₂CH₂O)₄C₆H₁₃ |
| O-47 | 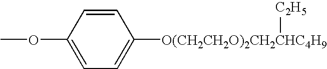 | 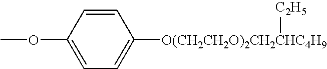 | —O—(CH₂CH₂O)₂C₆H₁₃ |
| O-48 | 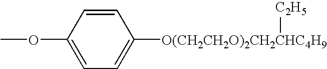 | 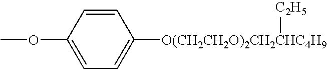 | 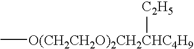 |
| O-49 | 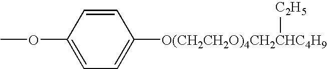 | 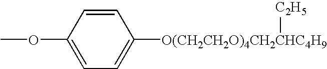 | 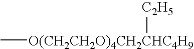 |
| O-50 | 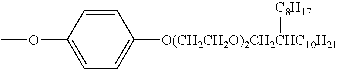 | 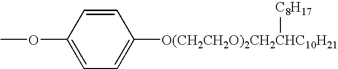 | —O—(CH₂CH₂O)₄C₆H₁₃ |
| O-51 | 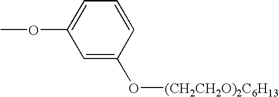 | 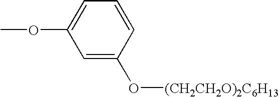 | —O—(CH₂CH₂O)₂C₆H₁₃ |
| O-52 | 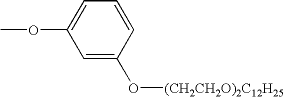 | 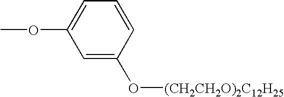 | —O—(CH₂CH₂O)₂C₆H₁₃ |
| O-53 | 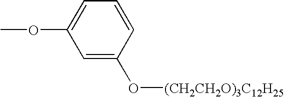 | 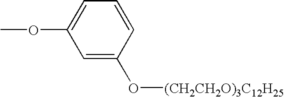 | —O—(CH₂CH₂O)₂C₆H₁₃ |
| O-54 | 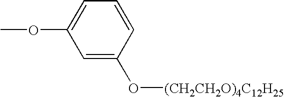 | 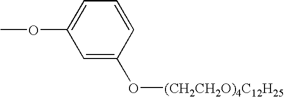 | —O—(CH₂CH₂O)₂C₆H₁₃ |
| O-55 | 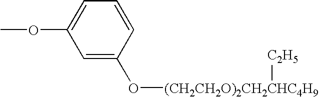 | 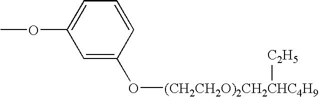 | —O—(CH₂CH₂O)₂C₆H₁₃ |
| O-56 | 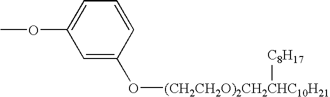 | 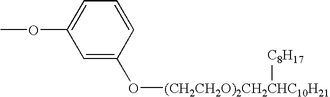 | —O—(CH₂CH₂O)₄C₆H₁₃ |
| O-57 | 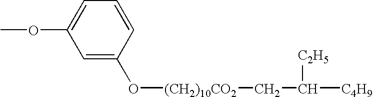 | 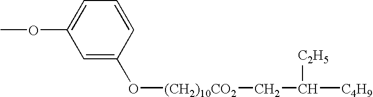 | —O—(CH₂CH₂O)₂C₆H₁₃ |
| O-58 | 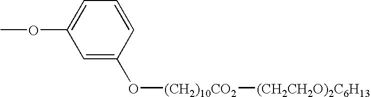 | 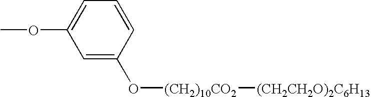 | —O—(CH₂CH₂O)₂C₆H₁₃ |
| O-59 | 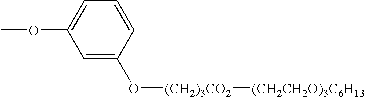 | 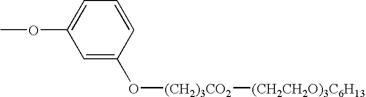 | —O—(CH₂CH₂O)₂C₆H₁₃ |
| O-60 | 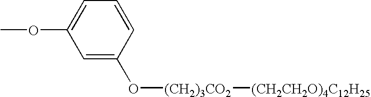 | 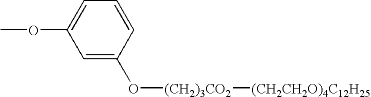 | —O—(CH₂CH₂O)₂C₆H₁₃ |
| N-1 | 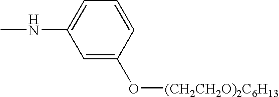 | 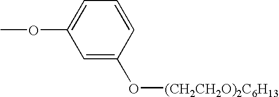 | —O—(CH₂CH₂O)₂C₆H₁₃ |
| N-2 | 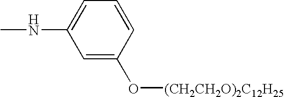 | 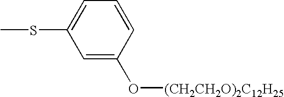 | —O—(CH₂CH₂O)₂C₆H₁₃ |
| N-3 | 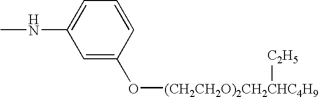 | 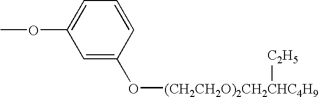 | —O—(CH₂CH₂O)₂C₆H₁₃ |
| N-4 | 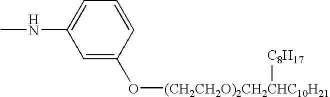 | 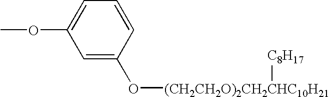 | —O—(CH₂CH₂O)₄C₆H₁₃ |
| N-5 | 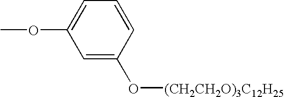 | 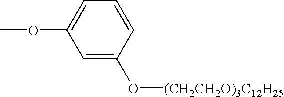 | 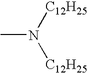 |
| N-6 | 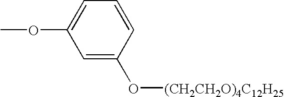 | 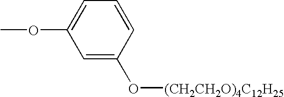 | 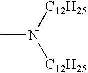 |
| N-7 | 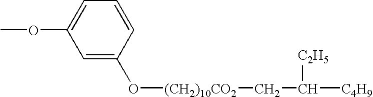 | 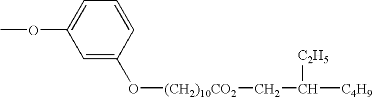 | 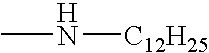 |
| N-8 | 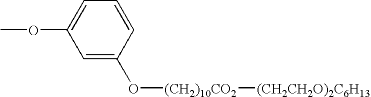 | 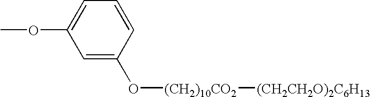 | 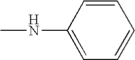 |
| N-9 | 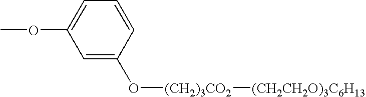 | 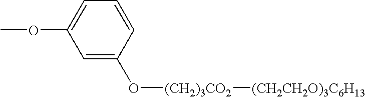 | —S—C₈H₁₇ |
| N-0 | 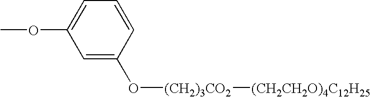 | 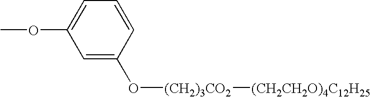 | —S—C₈H₁₇ |

The compounds represented by the formula (1) can be synthesized by using cyanuric chloride, which is readily commercially available, as a starting material. The compounds are preferably produced by the reaction of cyanuric chloride, which is to be a mother core, and a compound having an active hydrogen atom(s) such as a derivative of amine, aniline, alcohol, phenol, thioalcohol or thiophenol.

The reaction may be carried out in an organic solvent. Examples of the organic solvent which can be used for the reaction include halogenated-hydrocarbon organic solvents such as dichloromethane, ester organic solvents such as methyl acetate and ethyl acetate, ketone organic solvents such as acetone and methylethyl ketone, ether organic solvents such as tetrahydrofuran and dioxane, nitrile organic solvents such as acetonitrile and propionyl nitrile, amide organic solvents such as N,N-dimethyl formamide, N,N-dimetyl acetamide, 1,3-dimethyl-2-imidazolydone, 1,3-dimethyl-3,4,5,6,-tetrahydro-2(1H)— pyrimidinone (DMPU) and hexamethyl triamide phosphate, and sulfoxide organic solvents such as dimethylsulfoxide. Any catalyst and any base may be used in the reaction if necessary.

The compound represented by the formula (1), which can be employed in the invention, exhibits a minimum friction coefficient under a pressure equal to or greater than 10 MPa with the increase of a pressure and a viscosity-pressure coefficient equal to or less than 20 GPa¹ at 40° C. The compounds exhibiting a minimum friction coefficient under a pressure equal to or greater than 100 MPa are preferred. The minimum friction coefficient of the compound is preferably equal to or less than 0.07, and more preferably equal to or less than 0.05. It is known that, under a pressure equal to or greater than 10 MPa, the effect of elastic distortion begins to appear at various interfaces even of glasses or steels with the increase of the pressure. Accordingly, the composition of the invention is effective for a sliding part, which mainly operates under a pressure equal to or greater than 10 Mpa, preferably equal to or greater than 50 Mpa, and much more preferably equal to or greater than 100 MPa.

The lubricant composition may reach the mixed lubrication state along with the increase of pressure, and, then, the film interface may be broken. Accordingly, the composition of the invention may develop more effective friction coefficient, compared with conventional lubricant oils, with the increase of pressure ranging between a low of a pressure equal to or greater than 10 MPa and a high of a pressure under which the composition reaches the mixed lubricant state.

In usual, according to fluid lubrication operation, the generated pressure increases as the clearances are smaller. And, the pressure generated in concentrated contact such as point contact and line contact of ball bearings, gears or cams may range between a low of hundreds MPa and a high of GPa order. Therefore, in addition to elastic distortion of interfaces themselves, the viscosities of lubricant fluids increase depending on pressures exponentially. Under such a condition, the relationship between the pressure and the viscosity of the lubricant composition is expressed by BARUS formula as follows:
η=η₀ exp(αP) (1)

Logarithms of both sides are as follows:
logη=logη₀+loge×αP (2)

The relationship between logarithm of r and pressure P is a linear relationship with a slope α. The measure of depending property of viscosity on pressure is defined as viscosity-pressure coefficient.

The compound represented by the formula (1), which can be employed in the invention, exhibits a viscosity-pressure coefficient equal to or less than 20 GPa⁻¹ at 40° C. It is to be noted that a viscosity-pressure coefficient can be calculated according to a method described in “TRIBOLOGIST”, vol. 38, No. 10, pp. 927 (1993). The viscosity-pressure coefficient of the lubricant composition of the invention is preferably equal to or less than 13 GPa⁻¹ at 40° C.

For a compound being in a solid state at 40° C., a viscosity-pressure coefficient at 40° C. is defined as the value which is obtained by extrapolating from its viscosity-pressure coefficients at two or more temperatures at which the compound is in a liquid state

The apparent viscosity of the lubricant composition of the invention is preferably equal to or less than 300 mPa·S, more preferably equal to or less than 250 m Pa·S, and much more preferably equal to or less than 150 m Pa·S at 40° C. The lowest value of the apparent viscosity is generally, but not to be limited, 5 m Pa·S around. The lubricant composition, having an apparent viscosity at 40° C. falling within the above range, may exhibit appropriate lubricant properties even under a small pressure, and is preferred. It is to be noted that an apparent viscosity can be measured by using a common rotational viscometer, a viscosity/viscoelasticity measuring equipment or the like.

The lubricant composition may consist one or more compounds represented by the formula (1), or further comprise a base oil. The composition of the latter embodiment preferably comprises at least one compound represented by the formula (1) in an amount of 0.1 to 10 wt %, more preferably in an amount of 1 to 10 wt % and much more preferably in an amount of 1 to 5 wt %. The composition containing the compound in an amount falling within the above range is preferred in the view of improvements in ability of forming oil films and durability enhancement.

The composition of the invention may comprise a base oil. Any types of base oils may be employed in the invention, and the base oil may be selected from either mineral oils or synthetic oils. In the view of reduction of sludge, the base oil is preferably selected from synthetic oils and more preferably selected from synthetic carbon hydrate base oils. The composition, comprising, as base oil, at least one selected from the group consisting of poly-alpha-olefins, poly-alpha-olefin hydrates, ethylene-alpha-olefin copolymers, ethylene-alpha-olefin copolymer hydrates, mixtures of poly-alpha-olefin or hydrate thereof and alkyl naphthalene, mixtures of ethylene-alpha-olefin copolymer or hydrate thereof and alkyl naphthalene is preferred in the view of compatibility with the compound represented by the formula (1), reduction of sludge and durability enhancement.

Various types of poly alpha-olefin hydrates, referred to as “PAO” hereinafter, can be employed as base oil in the invention. In usual, PAO having a mean molecular weight of 200 to 1600 is preferred and PAO having a mean molecular weight of 400 to 800 is more preferred. Such PAO can be produced by hydrogenating the polymers which are produced by carrying out polymerization of 1-decene, isobutene or the like in the presence of catalyst such as Lewis acid complex or aluminum oxide catalyst. It is possible to improve durability of the composition and remarkably reduce the amount of sludge generating from the composition by employing such PAO as base oil.

Various types of ethylene-alpha-olefin copolymers, referred to as “PEAO” hereinafter, can be employed as base oil in the invention. PEAO may be produced by hydrogenating the polymers which are produced by carrying out polymerization of ethylene and alpha-olefin such as 1-decene and isobutene in the presence of catalyst such as Lewis acid catalyst. In usual, PEAO having a mean molecular weight of 200 to 4000 is preferred and PEAO having a mean molecular weight of 1000 to 2000 is more preferred.

The alkyl naphthalene, which can be employed in the invention, is selected from any naphthalene derivatives having one or more substituents on the naphthalene ring. Mono- di- or tri-alkyl naphthalenes, in which the total carbon atom number of the alkyl group(s) is from 5 to 25 around, are preferred; and, among these, naphthalenes having both of lower and higher alkyl groups are more preferred. Examples of the lower alkyl group include methyl, ethyl, propyl and isopropyl, and methyl is preferred. The higher alkyl group is not to be limited to a certain group, and may be selected from linear and branched chain alkyl groups. In the view of viscosity index or lubricant property, the higher alkyl group is preferably a linear chain alkyl group. Examples of such alkyl naphthalene include dialkyl naphthalenes having a methyl and a secondary C₁₀₋₂₄ alkyl group and mixtures thereof which are described in JPA No. hei 8-302371. Known materials, especially commercially available materials, are preferred in the view of procurement easiness.

As a base oil to be employed in the invention, mixtures of PAO or PEAO and alkyl naphthalene are preferred. As to the mix proportion thereof, the proportion of the former is preferably from 0.1 to 50 wt % and more preferably from 2 to 40 wt %; and the proportion of the later is preferably from 50 to 99.9 wt % and more preferably from 60 to 98 wt %. When the proportion of PAO or PEAO and alkyl naphthalene falls within the range, the durability and the ability of forming oil films can be improved.

The composition of the invention may comprise any known additives in order to attain practical performances adopted for the individual applications. Examples of the additive include wear preventive agents, extreme pressure agents, antioxidants, viscosity index raising agents, clean dispersion aids, metal passivation agents, corrosion preventive agents, rust preventive agents, and defoaming agents in an amount without lowering the effect of the invention.

The lubricant composition, with which a sintered body is impregnated, may be employed in at least a part of a sliding part of a bearing apparatus. The composition, for example, may be kept within pores of a porous sintered body. The composition is fed from the oil-impregnated sintered bearing disposed to a sliding site between a rotating element and a non-rotation body for bearing the rotating element, and contributes to reducing friction and wear.

The invention also relates to a bearing apparatus for bearing a rotating element rotatably comprising a sliding part wherein at least a part of the sliding part is a sintered body impregnated with the composition of the invention; and a sliding member comprising a sintered body impregnated with the composition of the invention. The porous sintered body may be used in the invention, and employing the porous sintered body, the lubricant composition of the invention may be kept within the pores of the porous sintered body. Any types of sintered bodies such as metal sintered bodies can be employed in the invention. Metal sintered bodies may be produced by sintering metal powders, comprising, as a major material, one or more types of metal powders selected from the common metal powders such as copper, iron and aluminum powder, and, if necessary, one or more types of powders selected from tin, lead, graphite and their alloy metal powders. It is possible to provide a long-life and stably operable bearing apparatus by employing the sintered body impregnated with the composition for a sliding part.

The bearing apparatus of the invention can be employed as a small size motor in the various technical fields such as automobiles, audio equipments, office equipments, home electric equipments and agricultural machines.

### EXAMPLES

The invention will be further specifically described below with reference to the following Examples. Materials, reagents, amounts and proportions thereof, operations, and the like as shown in the following Examples can be properly changed so far as the gist of the invention is not deviated. Accordingly, it should not be construed that the scope of the invention is limited to the following specific examples.

### Example of Synthesis of Compound S-7

Compound S-7 was synthesized according to the scheme shown below. 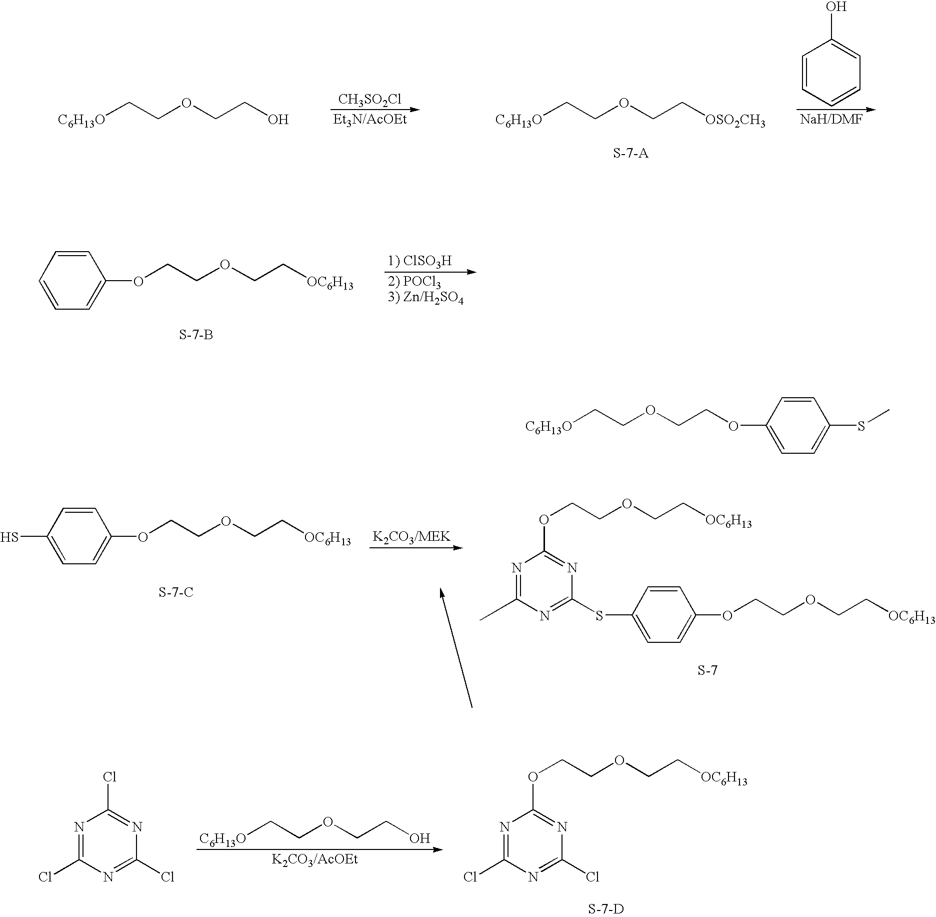
(Synthesis of Compound S-7-A)

In a 1 L three-necked flask provided with a stirrer, 190.28 g (1.0 mol) of diethylene glycol monohexyl ether, 250 mL of ethyl acetate and 121.0 mL (1.2 mol) of triethylamine were mixed under stirring to prepare a solution. The solution was cooled down to not higher than 5° C. and added dropwise with 120.2 g (1.05 mol) of methane sulfonyl chloride under stirring. Following the end of the addition, the solution was stirred at room temperature for two hours. Extracted with ethyl acetate and washed with water, the organic layer was isolated and dried with magnesium sulfate anhydride. After the removal of the solvent from the organic solution by the evaporation under a reduced pressure, 268.8 g of Compound S-7-A was obtained.

(Synthesis of Compound S-7-B)

In a 500 mL three-necked flask provided with a stirrer, 18.8 g (0.2 mol) of phenol and 150 mL of N,N-dimethyl formamide were mixed under stirring to prepare a solution. The solution was added with 8.8 g (0.22 mol) of sodium hydride (60% in oil) under stirring. The solution was added dropwise with 67.1 g (0.25 mol) of Compound S-7-A under stirring. Following the end of the addition, the solution was heated up to 100° C. and stirred for one hour. After cooled down to room temperature, extracted with ethyl acetate and washed with water, the organic layer was isolated and dried with magnesium sulfate anhydride. After the removal of the solvent from the organic solution by the evaporation under a reduced pressure and the purification of the product, 51.7 g (97%) of Compound S-7-B was obtained.

(Synthesis of Compound S-7-C)

In a 1 L three-necked flask provided with a stirrer, 45.3 g (0.17 mol) of Compound S-7-B and 70 mL of methylene chloride were mixed and stirred to prepare a solution. The solution was cooled down to −5° C. and added dropwise with a solution, prepared by dissolving 17.2 mL (0.25 mol) of chlorosulfonic acid in 20 mL of methyl chloride, under stirring. Following the end of the addition, the solution was stirred at room temperature for one hour. Then, the solution was cooled down to −10° C., added with 50 mL of acetonitrile and 50 mL of N,N-dimethyl acetamide, and stirred to prepare a solution. The solution was added dropwise with 44.0 g (0.29 mol) of phosphorous oxychloride. Following the end of the addition, the solution was stirred at room temperature for two hours. Extracted with ethyl acetate and washed with water, the organic layer was isolated and dried with magnesium sulfate anhydride. After the removal of the solvent from the organic solution by the evaporation under a reduced pressure, an intermediate, sulfonyl derivative, was obtained.

In a 1 L three-necked flask provided with a stirrer, 27 mL of conc. sulfuric acid and 80 mL of water were mixed, cooled down to −10° C., and added with the obtained sulfonyl derivative to prepare a solution. The solution was added with 45.6 g (0.7 mol) of zinc slowly. Then, the solution was refluxed with stirring under heating at 90° C. for two hours. The hot solution was filtered through sellite and washed with ethyl acetate. The filtrate was extracted with ethyl acetate and washed with water, and the organic layer was isolated and dried with magnesium sulfate anhydride. After the removal of the solvent from the organic solution by the evaporation under a reduced pressure, 53.22 g (94%) of Compound S-7-C was obtained.

(Synthesis of Compound S-7-D)

In a 200 mL three-necked flask provided with a stirrer and a reflux condenser, 120 mL of ethyl acetate and 18.4 g (0.1 mol) of cyanuric chloride were mixed and stirred to prepare a solution. The solution was added dropwise with 20 mL of an ethyl acetate solution of diethylene glycol monohexyl ether (20.9 g (0.11 mol)) at room temperature. Subsequently, the solution was added with 15.2 g (0.11 mol) of potassium carbonate, and stirred 50° C. for 20 hours. The solution was filtered through sellite and washed with ethyl acetate. The filtrate was extracted with ethyl acetate and washed with water, and the organic layer was isolated and dried with magnesium sulfate anhydride. After the removal of the solvent from the organic solution by the evaporation under a reduced pressure, 20.3 g (60%) of Compound S-7-D was obtained.

(Synthesis of Compound S-7)

In a 200 mL three-necked flask provided with a stirrer and a reflux condenser, 77.6 g (0.26 mol) of Compound S-7-C and 120 mL of methylethyl ketone were mixed and stirred to prepare a solution. The solution was added dropwise with 35 mL of methylethyl ketone solution of 33.8 g (0.1 mol) of Compound S-7-D. Subsequently, the solution was added with 35.9 g (0.26 mol) of potassium carbonate, and stirred 80° C. for 5 hours. The solution was cooled down to room temperature, extracted with ethyl acetate and washed with water, and the organic layer was isolated and dried with magnesium sulfate anhydride. After the removal of the solvent from the organic solution by the evaporation under a reduced pressure and the purification of the product, 60.3 g (70%) of Compound S-7 was obtained.

1H NMR (300 MHz CDCl₃): δ7.4-6.8(m, 8H), 4.3-3.35(m, 30H), 1.6-1.25 (m, 24H), 0.85(t, 9H)

### Example of Synthesis of Compound O-7

Compound O-7 was synthesized according to the scheme shown below. 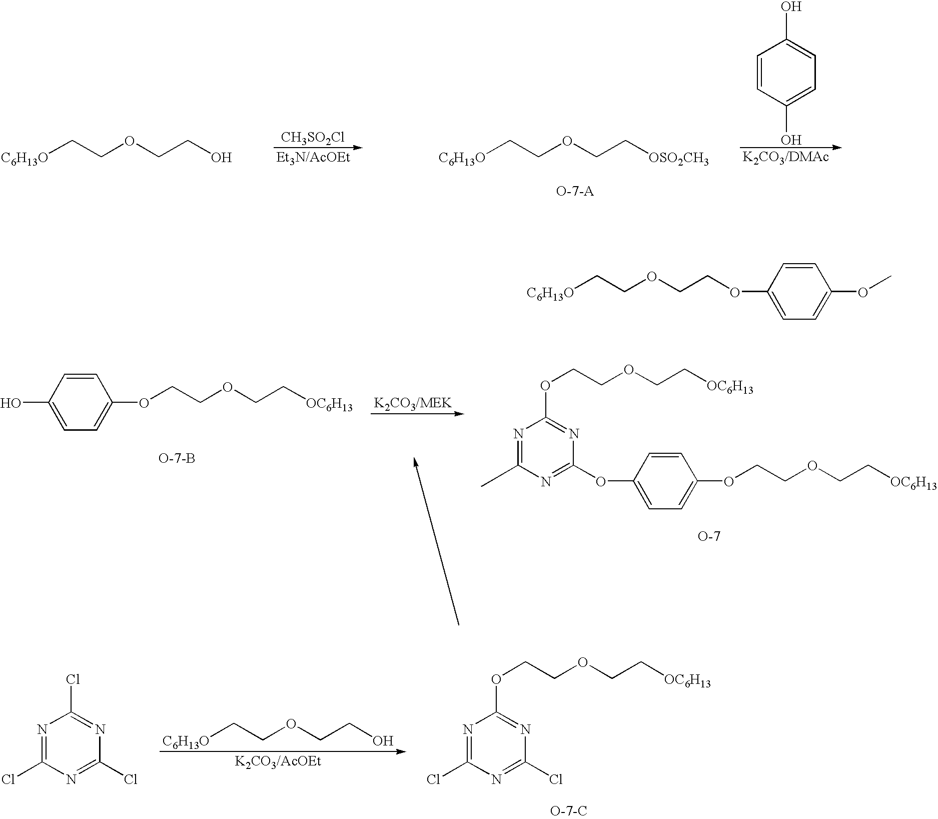
(Synthesis of Compound O-7-A)

In a 1 L three-necked flask provided with a stirrer, 190.28 g (1.0 mol) of diethylene glycol monohexyl ether, 250 mL of ethyl acetate and 121.0 mL (1.2 mol) of triethylamine were mixed under stirring to prepare a solution. The solution was cooled down to not higher than 5° C. and added dropwise with 120.2 g (1.05 mol) of methane sulfonyl chloride under stirring. Following the end of the addition, the solution was stirred at room temperature for two hours. Extracted with ethyl acetate and washed with water, the organic layer was isolated and dried with magnesium sulfate anhydride. After the removal of the solvent from the organic solution by the evaporation under a reduced pressure, 268.8 g of Compound O-7-A was obtained.

(Synthesis of Compound O-7-B)

In a 500 mL three-necked flask provided with a stirrer, 82.6 g (0.75 mol) of hydroquinone and 350 mL of N,N-dimethyl formamide and stirred to prepare a solution. The solution was added with 49.8 g (0.36 mol) of potassium carbonate under stirring. The solution was added dropwise with 80.5 g (0.3 mol) of Compound O-7-A under stirring. Following the end of the addition, the solution was heated up to 100° C. and stirred for two hours. The solution was cooled down to room temperature, extracted with ethyl acetate and washed with water, and the organic layer was isolated and dried with magnesium sulfate anhydride. After the removal of the solvent from the organic solution by the evaporation under a reduced pressure and the purification of the product, 50.8 g (60%) of Compound O-7-B was obtained.

(Synthesis of Compound O-7-C)

In a 200 mL three-necked flask provided with a stirrer and a reflux condenser, 120 mL of ethyl acetate and 18.4 g (0.1 mol) of cyanuric chloride were mixed and stirred to prepare a solution. The solution was added dropwise with 20 mL of an ethyl acetate solution of diethylene glycol monohexyl ether (20.9 g (0.11 mol)) at room temperature. Subsequently, the solution was added with 15.2 g (0.11 mol) of potassium carbonate, and stirred 50° C. for 20 hours. The solution was filtered through sellite and washed with ethyl acetate. The filtrate was extracted with ethyl acetate and washed with water, and the organic layer was isolated and dried with magnesium sulfate anhydride. After the removal of the solvent from the organic solution by the evaporation under a reduced pressure, 20.3 g (60%) of Compound O-7-D was obtained.

(Synthesis of Compound O-7)

In a 200 mL three-necked flask provided with a stirrer and a reflux condenser, 44.0 g (0.13 mol) of Compound O-7-C and 120 mL of methylethyl ketone were mixed and stirred to prepare a solution. The solution was added with 84.7 g (0.3 mol) of Compound Q-7-B. Subsequently, the solution was added with 45.6 g (0.33 mol) of potassium carbonate, stirred at room temperature for one hour, and, then, stirred under heating at 80° C. for 10 hours. The solution was cooled down to room temperature, extracted with ethyl acetate and washed with water, and the organic layer was isolated and dried with magnesium sulfate anhydride. After the removal of the solvent from the organic solution by the evaporation under a reduced pressure and the purification of the product, 77.8 g (70%) of Compound O-7 was obtained.

¹H NMR (300 MHz, CDCl₃): δ7.1-6.85(m, 8H), 4.4-3.4(m, 30H), 1.6-1.25(m, 24H), 0.85(t, 9H)

Although examples of syntheses of other compounds are not described in detail, it is possible to synthesize theme by referring to the above described examples of syntheses of Compound S-7 and O-7 and replacing the reagents with other reagents.

### Example Nos. 1 to 6

### Evaluation of Ability for Lubricant Composition

The friction coefficients of the compounds represented by the formula (1) were measured according to a friction test carried out under a condition described below. The friction coefficients of base oils (Comparative Example Nos. 1 to 4) were measured in the same manner as the compounds of the formula (1). It is to be noted that each friction coefficient was measured by using a reciprocating type friction test machine (SRV friction wear test machine) under a condition (i) or (ii) described below. The results of Example Nos. 1 to 6 are shown in Table 1, and the results of Comparative Example Nos. 1 to 4 are shown in Table 2.

It is to be noted that the apparent viscosities at 40° C. of the compositions, Example Nos. 1, 2, 4 and 5, were 167.0 mPa·s, 79.6 mPa·s, 90.3 mPa·s and 108.0 mPa·s respectively. All compounds employed in Example Nos. 1 to 6 were compounds exhibiting a viscosity-pressure coefficient equal to or less than 20 GPa⁻¹ at 40° C. and a minimum friction coefficient under a pressure equal to or greater than 10 MPa with the increase of a pressure.

Test Condition (i)

Tests were subjected under Cylinder on Plate Test.

Specimen (friction material): SUJ-2

Plate: 24 mm in diameter, 6.9 mm thick

Cylinder: 11 mm in diameter, 15 mm long

Temperature: 20° C.

Load: 400 N

Amplitude: 1.5 mm

Frequency: 50 Hz

Testing period: for 5 min. after the start of testing

Test Condition (ii)

Tests were subjected under Cylinder on Plate Test.

Specimen (friction material): SUJ-2

Plate: 24 mm in diameter, 6.9 mm thick

Cylinder: 11 mm in diameter, 15 mm long

Pretreatment: carrying out sliding preliminarily with 200 N load at 150° C. for 30 min.

Subsequently, a viscosity coefficient was measured under the condition described below.

Temperature: 60° C.

Load: 100N

Amplitude: 1.5 mm

Frequency: 50 Hz

Testing period: for 5 min. after the start of testing

TABLE 1

| | Example 1 | Example 2 | Example 3 | Example 4 | Example 5 | Example 6 |
|---|---|---|---|---|---|---|
| Discotic Compound | S-1 | S-7 | S-17 | S-33 | O-7 | O-24 |
| wt % | 100 | 100 | 100 | 100 | 100 | 100 |
| Base oil wt % | | | | | | |
| pentaerythritol | — | — | — | — | — | — |
| ester*1 | | | | | | |
| alkylbenzene*2 | — | — | — | — | — | — |
| naphthene base | — | — | — | — | — | — |
| mineral oil | | | | | | |
| paraffin base | — | — | — | — | — | — |
| mineral oil | | | | | | |
| SRV friction wear | 0.04 | 0.04 | 0.04 | 0.04 | 0.05 | 0.05 |
| test at 400 N, 20° C. | | | | | | |
| SRV friction wear | 0.03 | 0.03 | 0.03 | 0.03 | 0.04 | 0.04 |
| test at 100 N, 60° C. | | | | | | |

*1pentaerythritol hexanoate
*2alkylbenzene having a C₁₀ alkyl

TABLE 2

| | Comparative | Comparative | Comparative | Comparative |
|---|---|---|---|---|
| | Example 1 | Example 2 | Example 3 | Example 4 |
| Discotic | — | — | — | — |
| Compound | | | | |
| wt % | — | — | — | — |
| Base oil wt % | | | | |
| pentaerythritol | 100 | — | — | — |
| ester*1 | | | | |
| alkylbenzene*2 | — | 100 | — | — |
| naphthene base | — | — | 100 | — |
| mineral oil | | | | |
| paraffin base | — | — | — | 100 |
| mineral oil | | | | |
| SRV friction | 0.19 | 0.21 | 0.20 | 0.19 |
| wear test | | | | |
| at 400N, | | | | |
| SRV friction | 0.21 | 0.23 | 0.24 | 0.22 |
| wear test | | | | |
| at 100N, | | | | |

*1pentaerythritol hexanoate
*2alkylbenzene having a C₁₀ alkyl

From the results shown in the tables, it is understandable that the compositions embodying the invention exhibited a low friction coefficient under a high pressure relatively and exhibited a low friction coefficient even after being subjected to a high-temperature/high-pressure treatment.

The friction coefficients of a compound represented by the formula (2), Compound S-2, were measured under various pressures at 40° C. The results are shown in Table 3.

TABLE 3

| Plane Pressure | Friction Coefficient |
|---|---|
| 30 MPa | 0.180 |
| 46 MPa | 0.150 |
| 105 Mpa | 0.030 |
| 298 MPa | 0.035 |

(at 40° C.)

The viscosity-pressure coefficients of Compound S-2, were measured at various temperatures. The results are shown in Table 4.

TABLE 4

| | Viscosity-Pressure |
|---|---|
| Temperature | Coefficient (GPa⁻¹) |
| 20° C. | 16.8 |
| 40° C. | 12.1 |
| 50° C. | 10.3 |
| 80° C. | 8.96 |

### INDUSTRIAL APPLICABILITY

According to the invention, it is possible to provide a lubricant composition capable of exhibiting excellent properties not only in a state of mixture with conventional lubricant base oil, but also in a state not mixed with such lubricant base oil.

It is also possible to provide a lubricant composition capable of retaining low friction property and antiwearing property on the sliding surface for a long period, in particular even under extreme pressure.

It is also possible to provide a lubricant composition without environmentally-less-compatible heavy metals, phosphate group and sulfides to thereby concomitantly achieve both of longer service life and environmental compatibility.

It is also possible to provide a lubricant composition, exhibiting a low viscosity, excellent in evaporation characteristic.

It is also possible to provide a bearing apparatus which is long-life and is capable or working stably, and to provide a sliding member useful for a bearing apparatus.

What is claimed is:

1. A lubricant composition comprising at least one compound, exhibiting a minimum friction coefficient under a pressure equal to or greater than 10 MPa with the increase of a pressure and a viscosity-pressure coefficient equal to or less than 20 GPa⁻¹ at 40° C., represented by a formula (1): 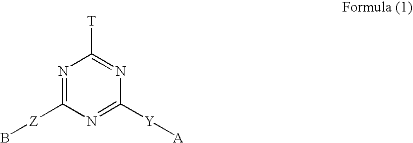
where Y and Z respectively represent a single bond or a bivalent linking group selected from the group consisting of NRa where Ra is a hydrogen atom or a C₁₋₃₀ alkyl group, oxygen, sulfur, carbonyl, sulfonyl and any combinations thereof; A and B respectively represent a substituted or non-substituted, alkyl group, alkenyl group, alkynyl group, aryl group or heterocyclic group; T is —S—R¹, —O—R² or —NR³R⁴; and R¹, R², R³ and R⁴ respectively represent a substituted or non-substituted, alkyl group, alkenyl group, alkynyl group, aryl group or heterocyclic group.

2. The lubricant composition of claim 1, exhibiting apparent viscosity equal to or less than 300 mPa·S at 40° C.

3. The lubricant composition of claim 1, wherein the compound represented by the formula (1) is a compound represented by a formula (2): 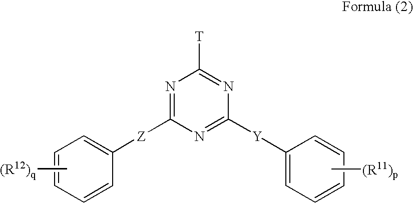
where Y and Z respectively represent a single bond or a bivalent linking group selected from the group consisting of NRa where Ra is a hydrogen atom or a C₁₋₃₀ alkyl group, oxygen, sulfur, carbonyl, sulfonyl and any combinations thereof; R¹¹ and R¹² respectively represent a substituent; T is —S—R¹, —O—R² or —NR³R⁴; R¹, R², R³ and R⁴ respectively represent a substituted or non-substituted, alkyl group, alkenyl group, alkynyl group, aryl group or heterocyclic group; and p and q respectively represents an integer from 1 to 5.

4. The lubricant composition of claim 1, wherein, in the formula (1), both of Y and z are sulfur atoms.

5. The lubricant composition of claim 1, wherein, in the formula (1), both of Y and z are oxygen atoms.

6. The lubricant composition of claim 1, wherein, in the formula (1), at least one of T, A and B contains an oligoalkyleneoxy group.

7. The lubricant composition of claim 1, to be used as an impregnating oil composition for a sintered bearing.

8. A bearing apparatus for bearing a rotating element rotatably comprising a sliding part wherein at least a part of the sliding part is a sintered body impregnated with a composition as set forth in claim 1.

9. A sliding member comprising a sintered body impregnated with a composition as set forth in claim 1.

10. A triazine-ring compound represented by a formula (2) 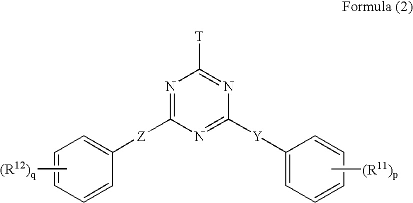
where Y and Z respectively represent a single bond or a bivalent linking group selected from the group consisting of NRa where Ra is a hydrogen atom or a C₁₋₃₀ alkyl group, oxygen, sulfur, carbonyl, sulfonyl and any combinations thereof; R¹¹ and R¹² respectively represent a substituent; T is —S—R¹, —O—R² or —NR³R⁴; R¹, R², R³ and R⁴ respectively represent a substituted or non-substituted, alkyl group, alkenyl group, alkynyl group, aryl group or heterocyclic group; and p and q respectively represents an integer from 1 to 5.

11. The compound of claim 10, wherein, in the formula (2), both of Y and Z are sulfur atoms.

12. The compound of claim 10, wherein, in the formula (2), both of Y and Z are oxygen atoms.

13. The compound of claim 10, wherein, in the formula (2), at least one of T, R¹¹ and R¹² represents a substituent containing an oligoalkyleneoxy chain.
